(19) United States
(12) Patent Application Publication
(10) Pub. No.: US 2022/0296674 A1
(43) Pub. Date: Sep. 22, 2022

(54) CELL PENETRATING PEPTIDES FOR INTRACELLULAR DELIVERY OF MOLECULES

(71) Applicants: INSERM (INSTITUT NATIONAL DE LA SANTÉ ET DE LA RECHERCHE MÉDICALE), Paris (FR); UNIVERSITÉ DE PARIS, Paris (FR)

(72) Inventors: Jean-Luc POYET, Paris (FR); Anne MARIE-CARDINE, Paris (FR); Justine HABAULT, Paris (FR); Claire FRASER, Paris (FR)

(21) Appl. No.: 17/625,006

(22) PCT Filed: Jul. 3, 2020
(86) PCT No.: PCT/EP2020/068790

§ 371 Date: Jan. 5, 2022

(30) Foreign Application Priority Data
Jul. 5, 2019 (EP) 19315060.4

(51) Int. Cl. A61K 38/08; A61K 47/64; A61P 35/00

(52) U.S. Cl. CPC A61K 38/08; A61K 47/645; A61P 35/00

(57) ABSTRACT

The inventors have identified a novel cell-penetrating sequence, termed hAP10, from the C-terminus of the human protein Acinus. hAP10 was able to efficiently enter various normal and cancerous cells, likely through an endocytosis pathway, and to deliver an EGFP cargo to the cell interior. Cell penetration of a peptide, hAP10DR, derived from hAP10 by mutation of an aspartic acid residue to an arginine was dramatically increased. Interestingly, a peptide containing a portion of the heptad leucine repeat region domain of the survival protein AAC-11 (residues 377-399) fused to either hAP10 or hAP10DR was able to induce tumor cells death in vitro and to inhibit tumor growth in vivo in a sub-cutaneous xenograft mouse model for the Sézary syndrome. Combined, the results indicate that hAP10 and hAP10DR may represent promising vehicles for in vitro or in vivo delivery of bioactive cargos, with potential use in clinical settings. Thus the present invention relates to cell penetrating peptides and uses thereof for intracellularly delivery of molecules.

### FIELD OF THE INVENTION

The present invention relates generally to the field of pharmaceutical sciences and, in particular, to the field of cell penetrating peptides.

### BACKGROUND OF THE INVENTION

The poor permeability and selectivity of the cell membrane strongly limit the repertoire of possible pharmaceutical agents and biologically active molecules. Established methods for delivery of cell-impermeable materials, such as viral vectors and membrane perturbation techniques, suffer a number of limitations, such as inefficiency, cytotoxicity or lack of reliability for in vivo settings (1,2). Consequently, in the recent years, much effort has been dedicated towards developing novel strategies allowing intracellular delivery of bioactive cargos into live cells. Cell-penetrating peptides (CPPs), also known as protein transduction domains (PTDs), are a class of short (less than 30 residues), cationic and/or amphipathic peptides which has been extensively shown to be capable of translocating though various biological membranes via direct penetration and/or endocytosis (3-6). Compared to other macromolecule carriers and enhancers of cellular entry, CPPs exhibits several advantages, such as usually low toxicity and rapid cellular internalization in a variety of cell types. Consequently, over the past few years, CPPs have received significant attention as delivery agents for a wide range of cargos such as proteins, peptides, DNAs, siRNAs, nanoparticles and small chemical compounds both in vitro and in vivo (7-11). Applications include both fundamental biology, such as transport of fluorescent or radioactive agents for imaging purposes, stem cell manipulation and reprogramming and gene editing (12-16), as well as preclinical and clinical trials to investigate medical applications of CPP-derived therapeutics against various diseases, including heart disease, stroke, cancer, and pain (see (7) for review). The promising results obtained in those studies highlight the potential of CPPs as an effective mean for intracellular molecular delivery. Most of the CPPs in use today are pathogen-derived or synthetic entities and therefore feature potential risk of immunogenicity and cytotoxicity, especially when conjugated to a protein or nanoparticle, restricting their use for biomedical applications (17,18). Moreover, many described CPPs exhibit low delivery efficiency. Consequently, the development of novel human-originated CPPs with a high transduction efficiency is of great interest.

### SUMMARY OF THE INVENTION

As defined by the claims, the present invention relates to cell penetrating peptides and uses thereof for intracellularly delivery of molecules.

### DETAILED DESCRIPTION OF THE INVENTION

The inventors have identified a novel cell-penetrating sequence, termed hAP10, from the C-terminus of the human protein Acinus. hAP10 was able to efficiently enter various normal and cancerous cells, likely through an endocytosis pathway, and to deliver an EGFP cargo to the cell interior. Cell penetration of a peptide, hAP10DR, derived from hAP10 by mutation of an aspartic acid residue to an arginine was dramatically increased. Interestingly, a peptide containing a portion of the heptad leucine repeat region domain of the survival protein AAC-11 (residues 377-399) fused to either hAP10 or hAP10DR was able to induce tumor cells death in vitro and to inhibit tumor growth in vivo in a sub-cutaneous xenograft mouse model for the Sézary syndrome. Combined, the results indicate that hAP10 and hAP10DR may represent promising vehicles for in vitro or in vivo delivery of bioactive cargos, with potential use in clinical settings.

Thus the first object of the present invention relates to a peptide that consists of the amino acid sequence as set forth in SEQ ID NO:1 (RSRSR-X6-RRRK wherein X6 is D or R).

In some embodiments, the peptide of the present invention consists of the amino acid sequence as set forth in SEQ ID NO:2 (RSRSRDRRRK) or SEQ ID NO:3 (RSRSRRRRRK).

As used herein, the terms “peptide,” “protein,” and “polypeptide” are used interchangeably to refer to a natural or synthetic molecule comprising two or more amino acids linked by the carboxyl group of one amino acid to the alpha amino group of another.

The peptides described herein can be prepared in a variety of ways known to one skilled in the art of peptide synthesis or variations thereon as appreciated by those skilled in the art. For example, synthetic peptides are prepared using known techniques of solid phase, liquid phase, or peptide condensation, or any combination thereof. Alternatively, the peptide of the present invention can be synthesized by recombinant DNA techniques well-known in the art. For example, the peptide of the present invention can be obtained as DNA expression products after incorporation of DNA sequences encoding for the peptide into expression vectors and introduction of such vectors into suitable eukaryotic or prokaryotic hosts that will express the desired peptide, from which they can be later isolated using well-known techniques.

A further object of the present invention relates to the use of the peptide of the present invention as a cell penetrating peptide.

As used herein, the term “cell-penetrating peptide” refers to a short peptide, for example comprising from 5 to 50 amino acids, which can readily cross biological membranes and is capable of facilitating the cellular uptake of various molecular cargos, in vitro and/or in vivo. The terms “cell-penetrating motif, “self cell-penetrating domain”, “cell-permeable peptide”, “protein-transduction domain”, and “peptide carrier” are equivalent.

A further object of the present invention thus relates to a method of transporting a cargo moiety to a subcellular location of a cell, the method comprising contacting the cell with the cargo moiety covalently linked to the peptide of the present invention for a time sufficient for allowing the peptide to translocate the cargo moiety to the subcellular location.

As used herein, the term “subcellular location” shall be taken to include cytosol, endosome, nucleus, endoplasmic reticulum, golgi, vacuole, mitochondrion, plastid such as chloroplast or amyloplast or chromoplast or leukoplast, nucleus, cytoskeleton, centriole, microtubule- organizing center (MTOC), acrosome, glyoxysome, melanosome, myofibril, nucleolus, peroxisome, nucleosome or microtubule or the cytoplasmic surface such the cytoplasmic membrane or the nuclear membrane.

As used herein, the term “cargo moiety” in its broadest sense includes any small molecule, carbohydrate, lipid, nucleic acid (e.g., DNA, RNA, siRNA duplex or simplex molecule, or miRNA), peptide, polypeptide, protein, bacteriophage or virus particle, synthetic polymer, resin, latex particle, dye or other detectable molecule that are covalently linked to the peptide directly or indirectly via a linker or spacer molecule. In some embodiments, the cargo moiety may comprise a molecule having therapeutic utility or diagnostic utility. Alternatively, the cargo moiety may a toxin or a toxin subunit of fragment thereof.

In some examples, the cargo moiety comprises a therapeutic moiety. Therapeutic moiety refers to a group that when administered to a subject will reduce one or more symptoms of a disease or disorder. The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body. Also, the therapeutic moiety can be capable of inducing and/or priming the immune system against potential pathogens. The therapeutic moiety can, for example, comprise an anticancer agent, antiviral agent, antimicrobial agent, anti-inflammatory agent, immunosuppressive agent, anesthetics, or any combination thereof. In other examples, the therapeutic moiety comprises a therapeutic protein. In some examples, the therapeutic moiety comprises a targeting moiety. The targeting moiety can comprise, for example, a sequence of amino acids that can target one or more enzyme domains. In some examples, the targeting moiety can comprise an inhibitor against an enzyme that can play a role in a disease.

A further object of the present invention relates to a complex wherein the peptide of the present invention is covalently linked to the cargo moiety.

In some embodiments, the peptide of the present invention is fused to at least one heterologous polypeptide so as to form a fusion protein.

As used herein, the term “fusion protein” refers to the peptide of the present invention that is fused directly or via a spacer to at least one heterologous polypeptide. According to the invention, the fusion protein comprises the peptide of the present invention that is fused either directly or via a spacer at its C-terminal end to the N-terminal end of the heterologous polypeptide, or at its N-terminal end to the C-terminal end of the heterologous polypeptide. As used herein, the term “directly” means that the (first or last) amino acid at the terminal end (N or C-terminal end) of the polypeptide is fused to the (first or last) amino acid at the terminal end (N or C-terminal end) of the heterologous polypeptide. In other words, in this embodiment, the last amino acid of the C-terminal end of said polypeptide is directly linked by a covalent bond to the first amino acid of the N-terminal end of said heterologous polypeptide, or the first amino acid of the N-terminal end of said polypeptide is directly linked by a covalent bond to the last amino acid of the C-terminal end of said heterologous polypeptide. As used herein, the term “spacer” refers to a sequence of at least one amino acid that links the polypeptide of the invention to the heterologous polypeptide. Such a spacer may be useful to prevent steric hindrances.

In some embodiments, the heterologous polypeptide is a fluorescent protein. Exemplary fluorescent proteins can include, but are not limited to, green fluorescent protein (GFP) or enhanced green fluorescent protein (EGFP) or AcGFP or TurboGFP or Emerald or Azami Green or ZsGreen, EBFP, or Sapphire or T-Sapphire or ECFP or mCFP or Cerulean or CyPet or AmCyanl or Midori-Ishi Cyan or mTFPl (Teal) or enhanced yellow fluorescent protein (EYFP) or Topaz or Venus or mCitrine or YPet or PhiYFP or ZsYellowl or mBanana or Kusabira Orange or mOrange or dTomato or dTomato-Tandem or AsRed2 or mRFPl or JRed or mCherry or HcRedl or mRaspberry or HcRedl or HcRed-Tandem or mPlum or AQ 143.

In some embodiments, the heterologous polypeptide is a cancer therapeutic polypeptide. As used herein, the term “cancer therapeutic polypeptide” refers to any polypeptide that has anti-cancer activities (e.g., proliferation inhibiting, growth inhibiting, apoptosis inducing, metastasis inhibiting, adhesion inhibiting, neovascularization inhibiting). Several such polypeptides are known in the art. (See. e.g., (Boohaker et al., 2012; Choi et al., 2011; Janin, 2003; Li et al., 2013; Sliwkowski and Mellman, 2013)).

In some embodiments, the peptide of the present invention is fused to an AAC-11 derivative polypeptide.

As used herein the term “AAC-11” has its general meaning in the art and refers to the antiapoptosis clone 11 protein that is also known as Api5 or FIF. An exemplary human polypeptide sequence of AAC-11 is deposited in the GenBank database accession number: Q9BZZ5 set forth as SEQ ID NO:4.

for AAC-11 Q9BZZ5
SEQ ID NO: 4
MPTVEELYRNYGLADATEQVGQHKDAYQVILDGVKGGTKEKRLAAQFIPK
FFKHFPELADSAINAQLDLCEDEDVSIRRQAIKELPQFATGENLPRVADI
LTQLLQTDDSAEFNLVNNALLSIFKMDAKGTLGGLFSQILQGEDIVRERA
IKFLSTKLKTLPDEVLTKEVEELILTESKKVLEDVTGEEFVLFMKILSGL
KSLQTVSGRQQLVELVAEQADLEQTFNPSDPDCVDRLLQCTRQAVPLFSK
NVHSTRFVTYFCEQVLPNLGTLTTPVEGLDIQLEVLKLLAEMSSFCGDME
KLETNLRKLFDKLLEYMPLPPEEAENGENAGNEEPKLQFSYVECLLYSFH
QLGRKLPDFLTAKLNAEKLKDFKIRLQYFARGLQVYIRQLRLALQGKTGE
ALKTEENKIKVVALKITNNINVLIKDLFHIPPSYKSTVTLSWKPVQKVEI
GQKRASEDTTSGSPPKKSSAGPKRDARQIYNPPSGKYSSNLGNFNYEQRG
AFRGSRGGRGWGTRGNRSRGRLY

In some embodiments, the peptide of the present invention is fused to:
an amino acid sequence ranging from the phenylalanine residue at position 380 to the leucine residue at position 384 in SEQ ID NO:4 or,
i) an amino acid sequence ranging from the phenylalanine residue at position 380 to the isoleucine residue at position 388 in SEQ ID NO:4 or,
an amino acid sequence ranging from the phenylalanine residue at position 380 to the leucine residue at position 391 in SEQ ID NO:4 or,
an amino acid sequence ranging from the tyrosine residue at position 379 to the leucine residue at position 391 in SEQ ID NO:4 or,
an amino acid sequence ranging from the glutamine residue at position 378 to the leucine residue at position 391 in SEQ ID NO:4 or,
an amino acid sequence ranging from the leucine residue at position 377 to the leucine residue at position 391 in SEQ ID NO:4 or,
an amino acid sequence ranging from the lysine residue at position 371 to the glycine residue at position 397 in SEQ ID NO:4 or,
an amino acid sequence ranging from the lysine residue at position 371 to the leucine residue at position 391 in SEQ ID NO:4 or,
an amino acid sequence ranging from the phenylalanine residue at position 380 to the threonine residue at position 399 in SEQ ID NO:4 or,
an amino acid sequence ranging from the lysine residue at position 371 to the threonine residue at position 399 in SEQ ID NO:4 or,
an amino acid sequence ranging from the leucine residue at position 377 to the threonine residue at position 399 in SEQ ID NO:4.

In some embodiments, the fusion protein of the present invention consists of the amino acid sequence as set forth in SEQ ID NO:5 (RSRSRDRRRKLQYFARGLQVYIRQLRLALQGKT) or SEQ ID NO:6 (RSRSRRRRRKLQYFARGLQVYIRQLRLALQGKT).

A further object of the present invention relates to a method of therapy in a subject in need thereof comprising administering to the subject a therapeutically effective amount of the complex of the present invention wherein the peptide of the present invention is covalently linked to a therapeutic moiety.

As used herein, the term “subject” denotes a mammal, such as a rodent, a feline, a canine, and a primate. Preferably a subject according to the invention is a human. Preferably a subject according to the invention is a subject afflicted or susceptible to be afflicted with a disease (e.g. a cancer).

In some embodiments, the complex of the present invention and in particular the fusion protein of the present invention is particularly suitable for the treatment of cancer.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is selected from the group consisting of breast cancer, triple-negative breast cancer, Acute Promyelocytic Leukemia (AML), hematologic cancer, lymphoma, B cell lymphoma, T cell lymphoma, B-cell non-Hodgkin's lymphoma, T-acute lymphoblastic leukemia, lung adenocarcinoma, kidney cancer, ovarian carcinoma, colon carcinoma, melanoma, Sezary syndrome.

A further object of the present invention relates to a pharmaceutical composition comprising the complex of the present invention (e.g. fusion protein) combined with pharmaceutically acceptable excipients, and optionally sustained-release matrices, such as biodegradable polymers, to form therapeutic compositions. As used herein the term “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. For instance, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

The peptide or the fusion protein of the invention may be formulated within a therapeutic mixture to comprise about 0.0001 to 1.0 milligrams, or about 0.001 to 0.1 milligrams, or about 0.1 to 1.0 milligrams, or about 1 to 10 milligrams or even about 10 to 100 milligrams per dose or so. Multiple doses can also be administered.

The invention will be further illustrated by the following figures and examples. However, these examples and figures should not be interpreted in any way as limiting the scope of the present invention.

### FIGURES

FIG. 1 Sequence and structural prediction of the investigated peptides. (A) Name, amino-acid sequences and support vector machine (SVM) score of the potential CPPs. The SVM-based method, which uses binary profile of the peptide, was used for the SVM score prediction. (B) Top: Structural prediction of hAP10 and hAp10DR. Bottom: Energy maps of hAP10 and hAP10DR. Coloring is the following: hydrogen donor favorable (yellow), hydrogen acceptor favorable (blue) and steric favorable (green).

FIG. 2 Cellular uptake of hAP10 and hAP10DR. (A) HUT78 cells were incubated with 5 μM of FITC-labelled hAP10 and hAP10DR or penetratin and TAT as controls for 1 h in complete medium. Cells were then washed with PBS, incubated in trypsin-EDTA solution (0.01% trypsin) at 37° C. for 10 min, resuspended in PBS and subjected to flow cytometry (right). Left: Bar diagram representing the uptake of the FITC-labelled peptides as mean cellular fluorescence from the flow cytometry analysis of live cells positive for FITC. Data are means±s.e.m. (n=3). (B) Fluorescence quantification of FITC-labelled hAP10 and hAP10DR uptaken in human B lymphocytes. Cells were incubated with 5 μM of FITC-labelled hAP10 and hAP10DR or penetratin and TAT as controls for 1 h in complete medium, washed with PBS and the fluorescence of the cell lysis measured as described in Material and Methods. Data are means±s.e.m. (n=3). (C) Intracellular distribution of FITC-labelled hAP10 and hAP10DR in U2OS cells. U2OS cells grown on coverslips were incubated with 5 μM of FITC-labelled hAP10 and hAP10DR or penetratin and TAT as controls for 1 h in complete medium, washed trice with PBS and live cells were imaged using fluorescence microscopy. All images were acquired using the same light intensity and microscope settings to permit direct comparison between the peptides.

FIG. 3 Internalization mechanisms of hAP10 and hAP10DR. C8161 cells pre-incubated at 4° C. or with heparin sulfate (20 μg/ml ), sodium azide (0.1%), CPZ (50 μM), MBCD (1 mM) or EIPA (50 μM) for 30 min or left untreated were incubated with 5 μM of FITC-labelled hAP10 and hAP10DR for 1 h in complete medium. Cells were then washed with PBS, detached with trypsin, washed and suspended in PBS, then subjected to flow cytometry (left). Right: Bar diagram representing the uptake of the FITC-labelled peptides as mean cellular fluorescence from the flow cytometry analysis of live cells positive for FITC. Data are means±s.e.m. (n=3).

FIG. 4 Lack of toxicity and immunogenicity of hAP10 and hAP10DR. (A) The indicated cells were exposed to increasing concentrations of hAP10 or hAP10DR for 20 h. Viability was then assessed by an MTT assay. Data are means±s.e.m. (n=3). (B) Necrotic cell death was monitored by lactate dehydrogenase (LDH) release from cells into the culture medium. The obtained values were normalized to those of the maximum LDH released (completely lysed) control. Data are means±s.e.m. (n=3). (C) hAP10 and hAP10DR do not induce hemolysis in vitro. Mice red blood cells were incubated with 30 μM of hAP10 or hAP10DR. Released hemoglobin was detected by densitometry at 540 nm. Hemoglobin release by cells treated with 1% Triton X-100 was used as 100% lysis control. (D) Levels of IL-6 secretion from RAW 264.7 cells exposed to 10 μM of hAP10 or hAP10DR or LPS (1 μg/ml) for 24 h. Data are means±s.e.m. (n=3).

FIG. 5 hAP10 and hAP10DR-mediated delivery of a GFP cargo into cells. (A) Electrophoretic analysis of the recombinant GFP derivatives. Samples (10 μg) of the indicated purified recombinant proteins were resolved by SDS-polyacrylamide gel electrophoresis followed by Coomassie Brilliant Blue staining. (B) U2OS cells were exposed to the indicated GFP fusion proteins (5 μM) for 1 h. Cells were then washed with PBS and live cells were imaged using fluorescence microscopy. All images were acquired using the same light intensity and microscope settings to permit direct comparison between the peptides.

FIG. 6 RT33 and RT33DR induces cancer cells, but not normal cells, death. (A) Amino-acid sequence of RT33 and RT3DR. hAP10 and hAP10DR sequences are in bold. (B) The indicated cells were exposed to increasing concentrations of RT33 or RT3DR for 20 h. Viability was then assessed by an MTT assay. Data are means±s.e.m. (n=3). (C) HUT78 cells were exposed to increasing concentrations of RT33 or RT3DR for 20 h in the presence and absence of 50 μM zVAD-fmk or 50 μM Necrostatin-1 (Nec-1). Viability was then assessed by an MTT assay. Data are means±s.e.m. (n=3). (D) HUT78 cells were exposed to 20 μM of RT33 or RT33DR for 3 h. Necrotic cell death was monitored by lactate dehydrogenase (LDH) as in FIG. 4 (B). Data are means±s.e.m. (n=3). (E) Ultrastructural analysis of HUT78 cells treated with 15 μM of hAP10 or hAP10DR for 1 h. (F) Structural prediction of RT33 and RT33DR. The segments corresponding to the hAP10 and hAP10DR moieties are in light grey. (G) Cancerous C8161 cells or non-cancerous MRC-5 cells were exposed to FITC-labelled RT33 or RT33DR for 1 h. Cels were then examined by fluorescence microscopy.

FIG. 7 RT33 and RT33DR specifically induce primary Sézary cells death. Sézary patients' PBMC were incubated with increasing concentrations of the indicated peptides for 4 h at 37° C. Cells were then analyzed by flow cytometry following labeling with anti-TCRVβ-FITC, -CD4-PE, -CD3-PE-Cy7 mAbs and 7-AAD. Data are shown as the means±s.e.m. of the percentage of 7-AAD+apoptotic cells within the following populations: malignant (CD3⁺CD4⁺TCR-V β⁺) and non-malignant (CD3⁺CD4⁺TCR-Vβ⁻) CD4+T-cells and non T-cells (CD3⁻), derived from three different patients.

FIG. 8 RT33 and RT33DR inhibit tumor growth in vivo in a mouse model for the Sézary syndrome. (A) Mice were engrafted subcutaneously with HUT78 Sézary cell line. Animals with preexisting tumors were treated daily with i.p. injections of RT33 or RT33DRM in normal saline (5 mg/kg) or normal saline as control. Tumors were calipered throughout the study and data were plotted as means±s.e.m. (n=7 mice per group). p<0.005 relative to control. Subsequently, tumors were excised, stripped of non-tumor tissue and tumors volumes were calculated. (B) Representative pictures of H&E staining of tumors treated with RT33, RT33DRM, or normal saline. The scale bar represents 500 μm.

### EXAMPLE

### Material & Methods

Peptides characterization

The support vector machine (SVM)-based prediction of cell penetrating properties was performed with the online CellPPD tool (25). Secondary structure predictions were performed with PSIPRED (28). Three-dimensional structure predictions were carried out with I-TASSER (29). Figures were generated with PyMOL (http://www.schrodinger.com). Energy maps of the peptides were analyzed and generated using Molegro Molecular Viewer.

Cellular uptake quantification

Cellular internalization of FITC-labelled peptides was analyzed using flow cytometry. Cells were incubated in the presence of the peptides (5 μM each) in complete medium for 1 h. Cells were then washed three times in PBS and incubated with trypsin (1 mg/ml) for 10 min to remove the extracellular unbound peptides. Finally, cells were suspended in PBS and kept on ice. FITC fluorescence intensity of internalised peptides in live cells was measured by flow cytometry using BD FACS CANTO II™ by acquiring 1×10⁴ cells. Data was obtained and analysed using FACSDIVA™ (BD biosciences) and FowJo software. In some experiments, cellular internalization was analysed using multimode spectrophotometry. Briefly, after incubation with the FITC-labelled peptides, cells were washed as described, centrifuged and the cell pellet resuspended in 300 μl of 0.1 M NaOH. Following 10 min incubation at room temperature, the cell lysate was centrifuged (14000 g for 5 min) and the fluorescence intensity of the supernatant determined (494/518 nm). The fluorescence of the cellular uptake is expressed as fluorescence intensity per mg of total cellular protein.

### Live Cell Microscopy

U2OS or C8161 cells (2×10⁴) were seeded into Lab-Tek II chamber slides (Nalgen Nunc, Rochester, N.Y.). 48 h latter, cells were incubated with either FITC-labelled peptides (5 μM) or the studied EGFP fusion recombinant proteins (5 μM) in complete medium for 1 h at 37° C. Following incubation, the cells were washed three times in PBS and imaged using a Zeiss Axiovert 200 M inverted fluorescence microscope.

Cell viability and lactate dehydrogenase (LDH) release assays

Cells survival was assessed with the CellTiter 96® Aqueous One Solution Cell Proliferation Assay kit (Promega, Madison, Wis.). Necrotic plasma membrane permeabilization was assessed by lactate dehydrogenase (LDH) leakage in the culture medium with the CytoTox 96® Non-Radioactive Cytotoxicity Assay kit (Promega, Madison, Wis.).

Hemolysis assay

Mice blood was centrifuged at 2000 rpm for 10 min. Red blood cell pellets were washed five times with PBS and resuspended in normal saline. For each assay, 1×10⁷ red blood cells were incubated with or without peptide (30 μM) in normal saline at 37° C. for 1 h. The samples were then centrifuged and the absorbance of the supernatant was measured at 540 nm. To determine the percentage of lysis, absorbance readings were normalized to lysis with 1% Triton X-100.

Immunogenicity assay

RAW 264.7 murine macrophages were seeded (1×104 cells/cm²) in a 24-well plate and allowed to grow for 24 h. Then, cells were left untreated or exposed to the hAP10 or hAP10DR peptides (10 μM) or to LPS (E. Coli O111:b4, 1 μg/ml) as a positive control for 24 h. Levels of IL-6 in the supernatants were analyzed using an Mouse IL-6 Quantikine ELISA Kit (R&D system).

Recombinant protein purification

TAT, penetratin, hAP10 and hAP10DR nucleotide sequences with EGFP inserted at the C-terminal end were subcloned in the pET-21a vector system (Novagen) and the constructs used to transform E.coli BL21(DE3) cells (Invitrogene). The transformed cells were grown at 37° C. in LB broth containing 100 ug/ml of ampicillin to an A₆₀₀ of 0.6 and induced with 1 mM IPTG for 3 h at 30° C. After harvest, the cells were resuspended in ice-cold Lysis buffer (20 mM HEPES, 100 mM NaCl, 10 uM ZNSO4, 1mM Tris-Hcl, pH 8.0) containing proteases inhibitors and lysed using a French press. Cell lysates were centrifuged at 4° C. for 30 min at 45000 rpm. Ni/NTA affinity purification was performed on an AKTA fast protein liquid chromatography (FPLC) system using 2 ml HisTrap HP columns (GE Healthcare Biosciences Uppsala, Sweden) equilibrated in wash buffer (20 mM HEPES, 100 mM NaCl, 10 uM ZNSO4, 1 mM Tris-Hcl, 20 mM imidazole, 10% glycerol, pH 8.0). Bound proteins were eluted using elution buffer B (20 mM HEPES, 100 mM NaCl, 10 uM ZNSO4, 1 mM Tris-Hcl, 300 mM imidazole, pH 8.0). Fractions were collected and analysed by Coomassie staining to assess purity.

Flow cytometry analysis of Sézary patients' cells

PBMC exposed or not to RT33 or RT33DR were processed for flow cytometry to assess cell death. Cells were labelled with a mix of anti-TCR-Vβ-FITC, -CD3-PE and -CD4-PECy7 mAbs (Beckman Coulter). Detection of apoptotic cells was performed using 7AAD (BD Biosciences). Cells were analyzed on a CytoFlex cytometer (Beckman Coulter) and data treated with FlowJo software.

Xenograft tumor model

Animal experiments were approved by The University Board Ethics Committee for Experimental Animal Studies (#2303.01). Xenograft tumors were obtained by subcutaneous injection of 10⁶ HUT78 cells in the right flank of 8-week-old female NOD-SCID-gamma (NSG) mice, bred and housed under pathogen-free conditions at our animal facility (IUH, Saint Louis Hospital, Paris, France). Treatment started after randomization when tumors were visible and consisted of daily intraperitoneal (i.p.) injection of normal saline or RT33 or RT33DR in normal saline (n=5 per group). Tumor volume was measured every other day and calculated as: long axis X short axis² ×0.5. Animals were euthanized after 21 days of treatment or when tumor size reached the ethical end point and visceral organs were excised for a gross pathological examination. Tumors were fixed in 4% neutral buffered formalin and embedded in paraffin. Sections (4 μm) were stained with hematoxylin-eosin (H&E) and subjected to microscopic analysis.

### Results

Acinus contains a CPP-like sequence

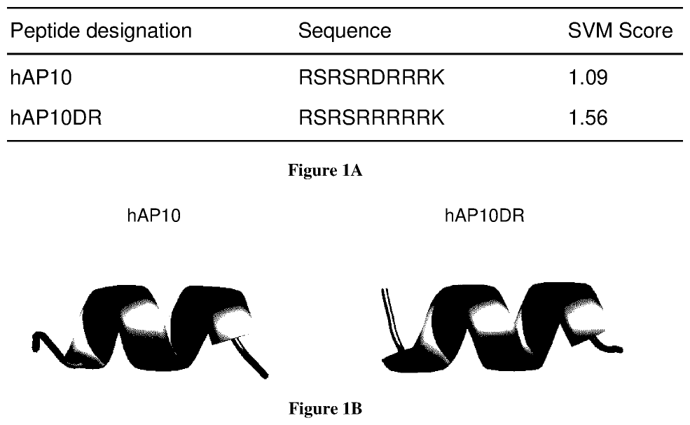

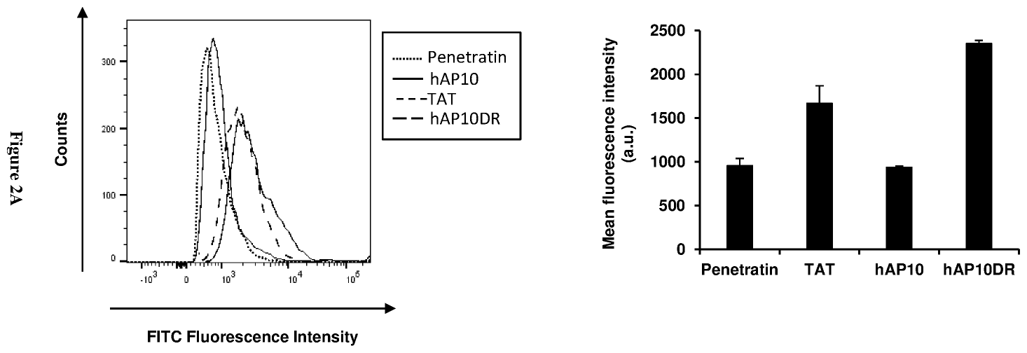

In exploring the sequence of Acinus (Apoptotic chromatin condensation inducer in the nucleus), a nuclear protein involved in in RNA processing and apoptotic DNA fragmentation (19-24), we noticed an arginine rich region located in the C-terminus that presents significant similarities with the sequence of the TAT CPP (residues 1177-1186 of Acinus-L, FIG. 1A). Analysis of this 10 residues sequence, hereafter called hAP10, using the CellPPD in silico tool (25) confirmed that hAP10 could indeed possess CPP properties (FIG. 1A). Cationic CPPs have a net positive charge at physiological pH, mostly derived from arginine and lysine residues in their sequence, which drives their cell-penetrating properties (7). hAP10 is highly cationic with six arginine and one lysine residues. As it contains one aspartic acid at its center, and because replacing negative charged residues with positively charged residues can increase penetrating activity of cationic CPPs (26), we wondered whether substitution of hAP10 aspartic acid to an arginine (hAP10DR) would potentially increase its penetrating properties. Indeed, as shown in FIG. 1A, CellPPD analysis resulted in a higher SVM score for hAP10DR compared to hAP10. Secondary structure of CPPs are important for their membrane interaction and it has been shown that peptides with an α-helical region can internalize more efficiently than their random-coiled counterparts (27). Secondary and three-dimensional structure predictions carried out with the well-established PSIPRED and I-TASSER servers (28,29) suggested an essentially helical structure for both hAP10 and hAP10DR, with an helical content of 70% and 80%, respectively (FIG. 1B). As these observations suggest that hAP10 and hAP10DR could both represent novel CPPs, both peptides were selected for experimental validation and further analysis of their in vitro and in vivo cargo delivery properties.

Cellular uptake of hAP10 and hAP10DR.

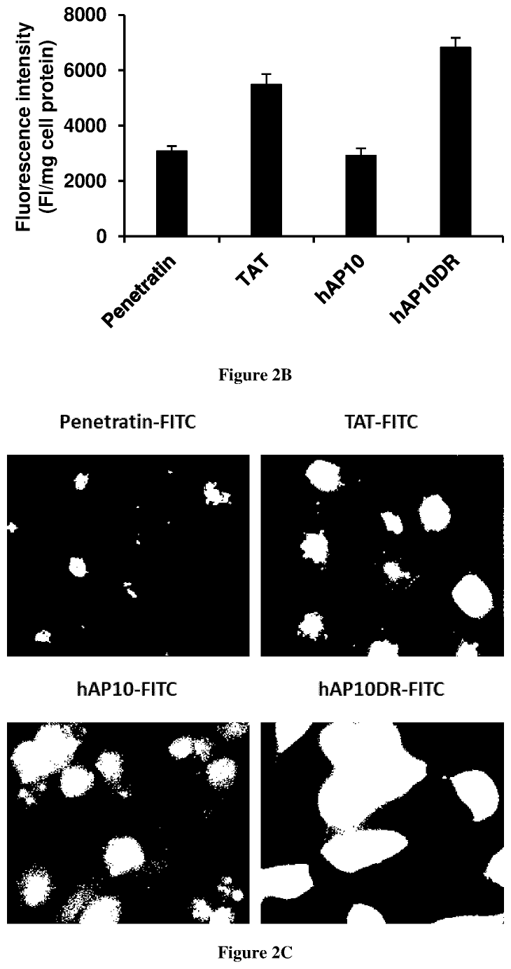

The translocation efficacy of FITC-labeled hAP10 and hAP10DR was first assessed by flow cytometry analysis and compared to that of the widely used CPPs penetratin and TAT. Cellular uptake was analyzed after 60 min incubation of HUT78 cells and stringent washing followed by incubation with trypsin to remove the extracellular membrane-associated peptides (5). As shown in FIG. 2A, both hAP10 and hAP10DR were efficiently internalized into HUT78 cells. Importantly, hAP10 displayed similar uptake efficiency to that of penetratin. hAP10DR however, showed a higher uptake and was internalized approximately twice as more efficiently than its wild type counterpart and about 50% more than TAT (FIG. 2A), indicating that replacement of the negatively charged aspartic acid with the positively charged arginine drastically favored the CPP capacities of the peptide. Similar data were obtained using U2OS and C8161 cancer cells (not shown). Interestingly, hAP10 and hAP10DR were able to permeate into non-cancerous cells, such as human B lymphocytes (FIG. 2B). We next examined the cellular distribution of hAP10 and hAP10DR using fluorescent microscopy imaging. U2OS cells were treated with FITC-labeled hAP10 and hAP10DR or the control peptides penetratin and TAT and the cells were imaged using live microscopy imaging. We chose to perform these experiments on live cells to avoid fixation artefacts that can arise when studying transduction of arginine-rich peptides (5). As shown in FIG. 2C, both hAP10 and hAP10DR as well as the control peptides adopted both a diffuse and punctuate fluorescence distribution throughout the cells, confirming that the peptides were indeed internalised and not merely adsorbed at the cell surface. In agreement with the cytometry profiles, the intracellular fluorescence intensity of the hA10DR peptide was much higher to that of hAP10 and control peptides penetratin and TAT, confirming the superior transduction efficacy of the mutated version of the peptide.

### Cellular uptake mechanism of hAP10 and hAP10DR.

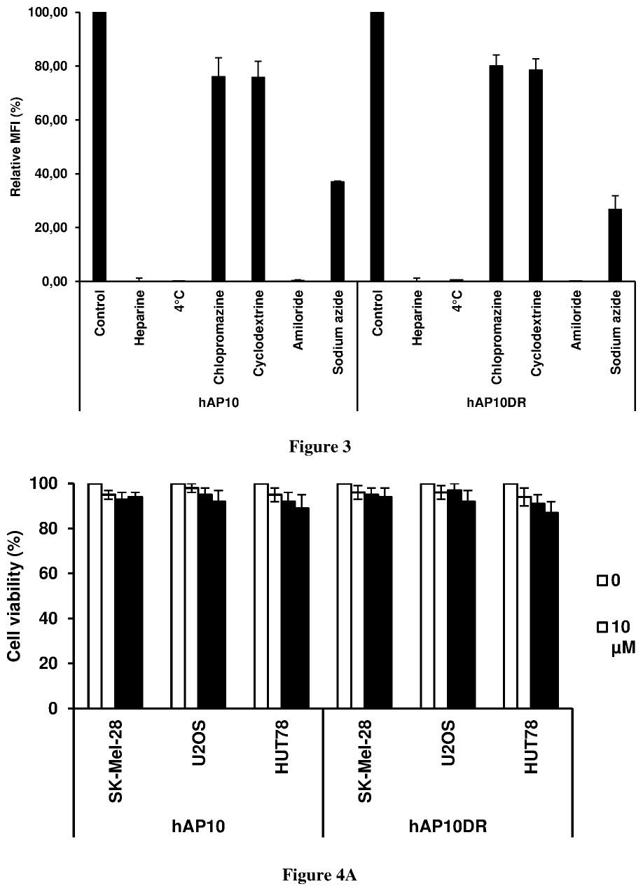

Although the precise mechanisms by which CPPs enter the cells are still under debate, they fall into two broad categories: direct translocation and endocytosis (7). To gain insight into the transduction process of hAP10 and hAP10DR, we investigated the effect of heparin, temperature and well-established endocytosis inhibitors on the cellular uptake of hAP10 and hAP10DR. As shown in FIG. 3, cellular uptake of both hAP10 and hAP10DR into C8161 cells was greatly decreased in the presence of heparin sulfate, indicating that the peptides penetrate the membrane via heparin sulfate proteoglycan (HSPG)-mediated pathway(s). Similar data were obtained using U2OS cells (not shown). We next tested whether the cellular internalization of hAP10 and hAP10DR was mediated by an energy-dependent process. As endocytosis is form of active transport, requiring energy, lowering the temperature is expected to inhibit endocytic processes but not energy-independent processes such as direct penetration. As shown in FIG. 3, cellular uptake of hAP10 and hAP10DR was substantially decrease when C8161 cells were incubated at 4° C. as compared to 37° C. Similar results were observed following energy depletion by sodium azide (FIG. 3). Combined, these data indicate that hAP10 and hAP10DR are internalized into cells through an energy-dependent endocytosis mechanism. We next evaluated the precise cell entry pathway of hAP10 and hAP10DR by using various inhibitors of known endocytic pathways. Pre-treatment of cells with chlorpromazine (CPZ), a known inhibitor of clathrin-mediated endocytosis, or methyl-β-cyclodextrine (MBCD), an inhibitor of lipid raft-mediated endocytosis, did not significantly reduced the uptake of hAP10 and hAP10DR (FIG. 3). However, a drastic decrease was observed upon pre-treatment of the cells with 5-(N-ethyl-isopropyl) amiloride (EIPA), an inhibitor of micropinocytosis (FIG. 3). Similar data were obtained when using U2OS cells (not shown). Together, these results identify macropinocytosis as the main pathway for hAP10 and hAP10DR cellular uptake.

Analysis of cellular toxicity, hemolytic activity and immunogenicity of hAP10 and hAP10DR.

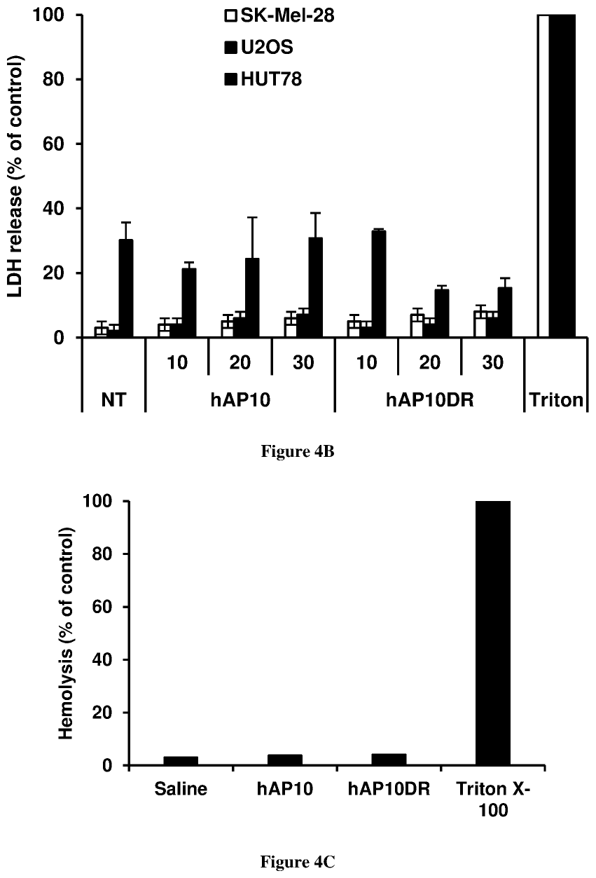

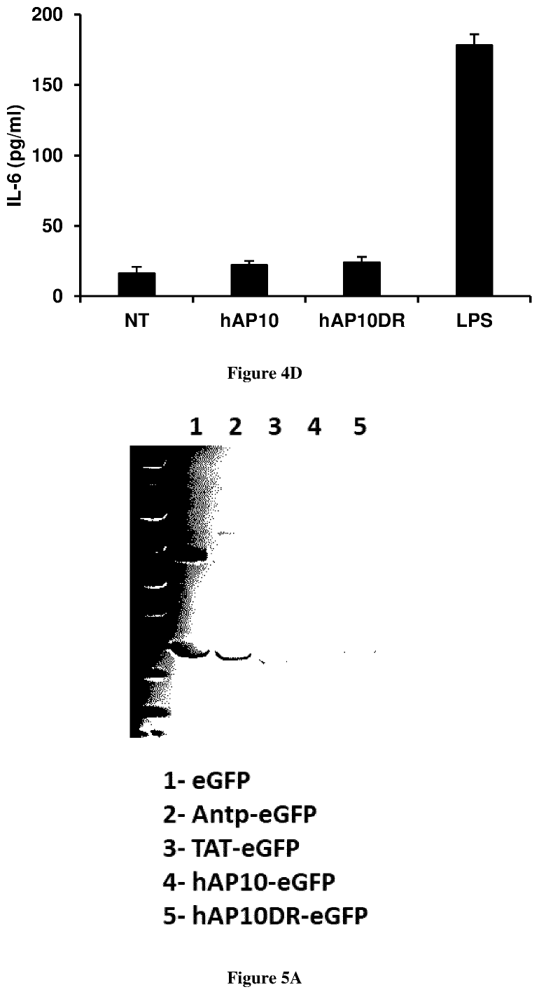

Similarly to other drug delivery systems, cytotoxicity and the tendency to induce innate immunity may limit CPPs uses in clinics. We first assayed the cytotoxicity effect of hAP10 and hAP10DR on various cell lines. Dose-response analyses indicate that neither peptide significantly altered cellular viability at doses up to 30 μM (FIG. 4A). Moreover, absence of lactate dehydrogenase (LDH) activity release in the culture medium indicated that hAP10 and hAP10DR did not induce membrane disturbance (FIG. 4B). In lane with this observation, neither hAP10 nor hAP10DR exhibited hemolytic activity (FIG. 4C), confirming that the peptides do not cause membrane damage. We next evaluated the potential immunogenicity of hAP10 and hAP10DR by measuring the secreted levels of IL-6 upon treatment of RAW 264.7 mouse macrophage cells with the peptides for 24 h. As shown in FIG. 4D, whereas the control bacteria-derived lipopolysaccharide (LPS) elicited a potent cytokine response, no significant IL-6 release was detected in the media of RAW 264.7 cells cultured in the presence of hAP10 or hAP10DR. Combined, our data indicate that hAP10 or hAP10DR are essentially not cytotoxic and non-immunogenic and therefore demonstrate potential for in vivo applications.

Intracellular delivery of hAP10- and hap10DR-GFP fusion protein.

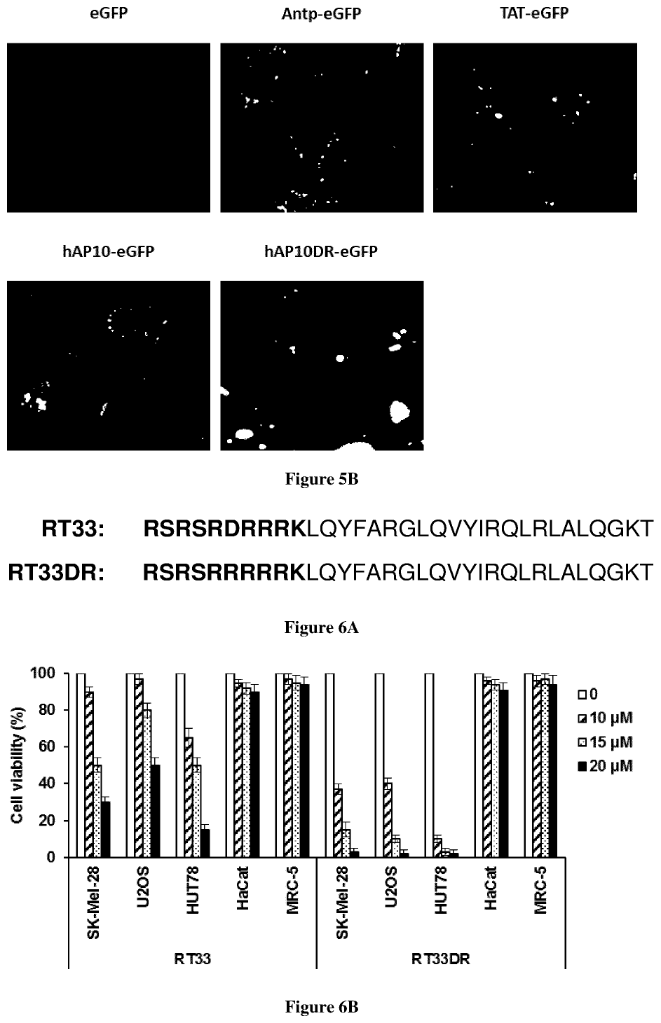

We next evaluated the potential of hAP10 and hAP10DR to carry a functional macromolecule into cells. For that purpose, we generated recombinant fusion proteins comprising EGFP fused at the N-terminus to hAP10 or hAP10DR or the control CPPs TAT and penetratin (FIG. 5A). The resulting proteins were then administered to the culture media of U2OS cells and the cells were imaged using live microscopy imaging. As shown in FIG. 5B, a punctate fluorescence pattern was observed for the fusions protein but not for EGFP alone. Interestingly, in lane with the FITC-labeled peptide uptake, hAP10-EGFP fluorescence was at least comparable to that of TAT-EGFP or penetratin-EGFP whereas hAP10DR-EGFP fluorescence was significantly higher. Taken together, our data indicate that the hAP10 and mutated sequences possess strong cell penetrating activities and are at least as effective as the commonly used TAT and penetratin CPPs at delivering an EGFP cargo to the cell interior.

Anti-tumoral effect of AAC-11 heptad leucine repeat-derived peptides.

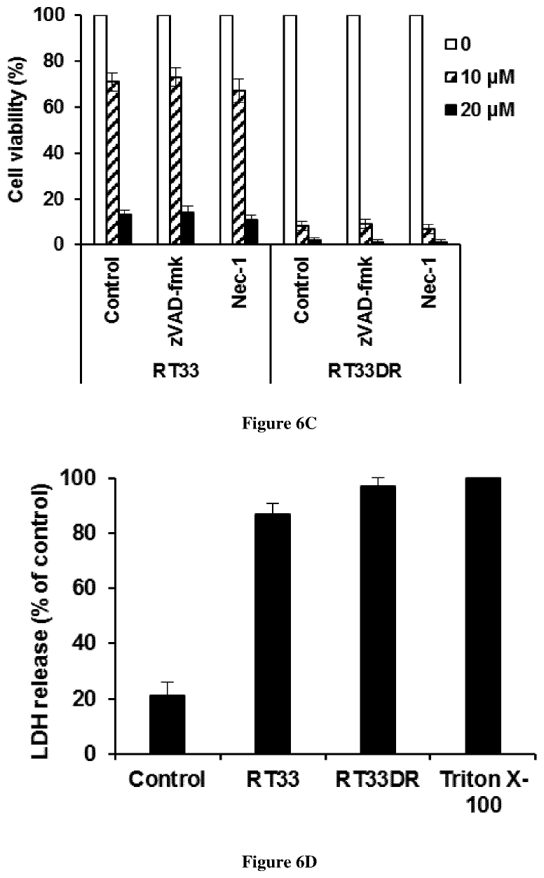

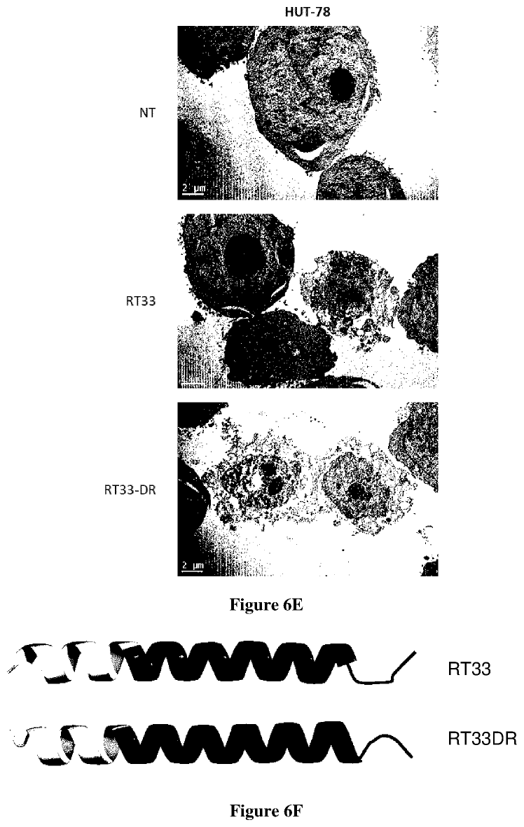

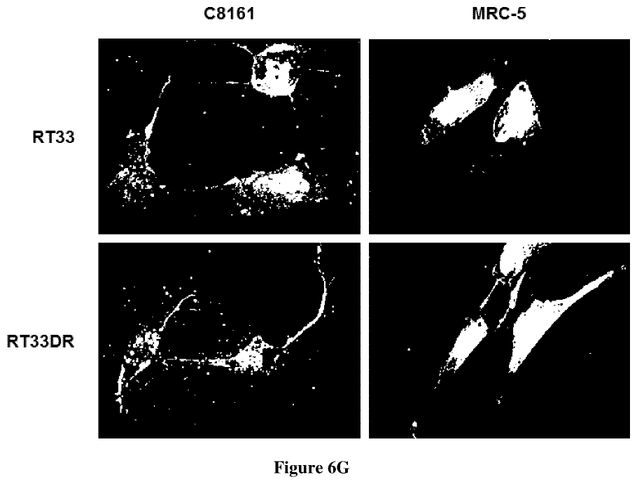

We have previously reported that a penetrating peptide (peptide RT53) spanning the heptad leucine repeat region of the survival protein AAC-11 (residues 363-399) fused to the CPP penetratin induces cancer cell death in vitro and inhibits melanoma tumor growth in a xenograft mouse model (30). We here hypothesized that a peptide comprising a smaller portion of the heptad leucine repeat region of AAC-11 attached to hAP10 or hAP10DR might possess interesting anti-cancer properties. We therefore tested the anti-tumor effects of shorter peptides containing AAC-11 residues 377-399 attached to the C-terminus of hAP10 or hAP10DR (RT33 and RT33DR peptides, respectively). To study the anticancer properties of the developed peptides, we first assessed the viability of various cancer or normal cells following exposure to increasing concentration of RT33 or RT33DR. As shown in FIG. 6A, both peptides inhibited cell viability in all cancer cells (SK-Mel-28, U2OS, HUT78 ) in a dose-dependent manner, while sparing the normal cells tested (HaCat, MRC-5). Of note, RT33DR exhibited substantially higher anticancer proprieties than RT33, maybe due to the high cell penetration capacity of its CPP. Neither the shuttles (FIG. 4) nor the AAC-11 specific portion alone (not shown) decreased cell viability, indicating that the integrity of the peptides is required for their anti-tumoral effects. We next sough to investigate RT33 and RT33DR mechanisms of cancer cell death. We were especially interested in the response of HUT78 Sézary cells because effective therapeutic options for Sézary syndrome, an erythrodermic form of cutaneous T-Cell lymphoma (CTCL), are scarce (31). Pharmacological inhibition of the apoptotic pathways with the pan-caspase inhibitor zVAD-fmk did not block RT33 or RT33DR-induced cytotoxicity (FIG. 6B), suggesting that the observed cell death does not depend on apoptosis. Furthermore, cell death was not prevented by the RIPK1 kinase inhibitor necrostatin-1, excluding necroptosis as cell death mechanism (FIG. 6C). Similar data were obtained using U2OS, C8161 and SK-MEL28 cells (not shown). In previous studies, we found that RT53 induces tumor cell necrosis, as evidenced by the rapid release of lactate dehydrogenase (LDH) from treated cancer cells (30). We therefore assessed LDH activity release in the culture medium of HUT78 cells treated with RT33 and RT33DR. As shown in FIG. 6D, peptides exposure resulted in a massive release of LDH into HUT78 treated cells supernatant, indicative of membrane lysis and necrotic cell death. Transmission electron microscopy micrographs further supported that RT33 and RT33DR induce tumor cell necrosis. Whereas control cells showed a typical intact outer plasma membrane, HUT78 cells treated with RT33 and RT33DR exhibited ruptured and disintegrated plasma membranes, with total loss of membrane structure (FIG. 6E). In line with our precedent results (FIG. 6C), no evidence of chromatin condensation was observed, indicating the RT33- and RT33DR-mediated cell death does not involve a direct form of conventional apoptosis but rather a membranolytic mode of action. Combined, our data indicate that like RT53, RT33 and RT33DR induce necrosis of cancerous cells. The ability of RT33 and RT33DR to induce plasma membrane leaking suggests that both peptides target the plasma membrane. Previous data obtained with RT53 peptide suggested that, in analogy with pore-forming toxins, its membranolytic property was a consequence of its accumulation at the plasma membrane of cancerous cells, leading to the formation of pore and subsequent necrosis (30). In this mechanism, the cell-penetrating moiety of RT53 allows its plasma membrane penetration, where it can bind to a membrane protein partner through its AAC-11 sequence. Local accumulation of the peptide would then lead to pores formation, owning to its alpha helical membrane active structure (30). Structure prediction indicated that, like RT53, RT33 and RT33DR should essentially adopt an α-helical structure (FIG. 6F). To provide evidence that RT33 and RT33DR target the plasma membrane, we incubated C8161 cells with FITC-labeled peptides and observed the fluorescence pattern. We chose C8161 cells as they are adherent and provide a big cytoplasm, which makes this cell line appropriate for imaging. As shown in FIG. 6G, RT33 and RT33DR treated cells showed punctate fluorescence over the cell surface, indicating that the peptides accumulate both at the plasma membrane and at the intracellular level. However, no RT33 or RT33DR fluorescence was observed in the membranes of the non-cancerous MRC-5 cells. Combined, our results strongly suggest that RT33 and RT33DR, owning to the cell-penetrating properties of the hAP10 and hAP10DR shuttles, can insert into cancer cells plasma membrane where the peptides, upon binding to a membrane-interacting partner, induce pore formation, as witnessed for the RT53 peptide.

RT33 and RT33DR induce targeted killing of circulating malignant T cells in Sézary patients' primary PBMC.

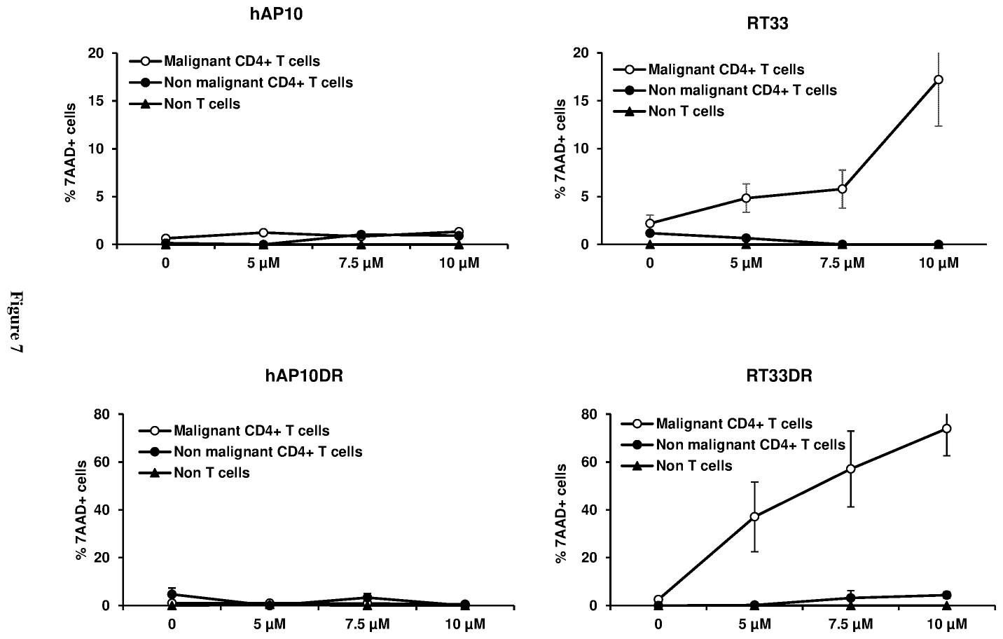

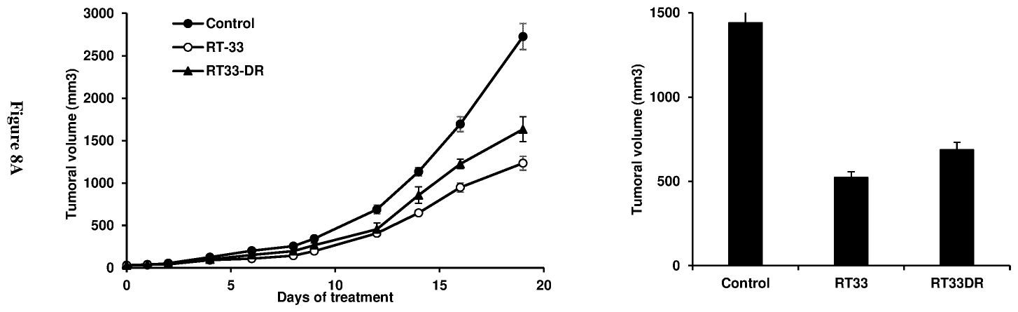

We next tested the anti-tumor effect of RT33 and RT33DR against primary Sézary cells. For that purpose, an ex vivo assay was established in which RT33 or RT33DR were directly incubated with peripheral blood mononuclear cells (PBMC) from Sézary patients. The viability of three different cell populations was then assessed by flow cytometry through the incorporation of 7-AAD : the malignant T-cell clone (Sézary cells), defined as CD3⁺CD4⁺Vβ⁺cells, the non-malignant CD4⁺T-cells, defined as CD3⁺CD4⁺Vβ⁻cells, and the non T-cells, defined as CD3⁻cells. As shown on FIG. 7 (right), both RT33 and RT33DR exhibited dose-dependent cell death activity in the malignant CD4⁺T-cells, RT33DR being the most efficient peptide toward Sézary cells. Strikingly, neither peptide decreased cell viability of the non-tumoral CD4⁺T-cell as well as non-T cell populations even at the highest doses. Therefore, these results demonstrate that RT33 and RT33DR selectively induce primary Sezary cells death in a dose-dependent manner, without harming primary normal cells, indicating that the peptides possess a cancer cell selective killing property. Finally, in lane with our previous data, the hAP10 or hAP10DR shuttles did not induce cell death in the transformed or normal primary cell populations (FIG. 7, left), confirming their safety profile as carrier.

RT33 and RT33DR induce tumor growth reduction in a xenograft murine model of Sézary syndrome.

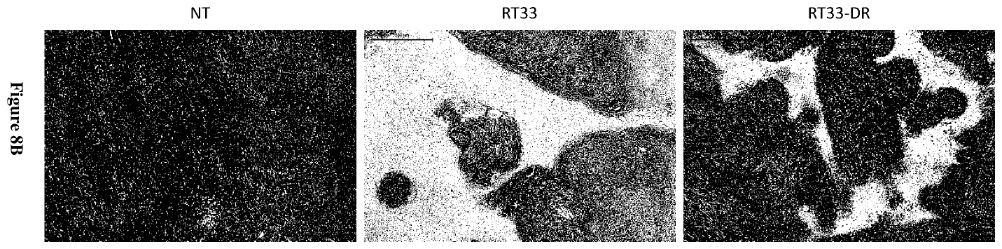

To assess in vivo antitumor activity of RT33 and RT33DR, HUT78 Sézary cells were inoculated subcutaneously to NOD/SCID gamma (NSG) mice. When the xenografted tumors reached a volume of approximately 100 mm³, mice were randomized and injected daily with normal saline (NT) or 5 mg/kg of RT33 or RT33DR peptides. No obvious clinical symptoms were observed during the experimental period with either peptide (not shown). As shown in FIG. 9A (left), both peptides induced significant tumor growth reduction as compared to control mice, with approximate tumor growth reduction of 66% (p<0.005) for RT33 and 60% for RT33DR. Similarly, upon sacrifice at the study end point, xenograft tumors were excised and stripped of non-tumor tissue, if present, for more precise ex vivo measurement. As shown in FIG. 8A (right), total tumor volume was decreased more than 2.6 times in RT33 treated mice and more than two fold in RT33DR treated mice as compared with that in control mice. Assessment of tumor necrosis by H&E staining revealed a sharp increase of necrotic areas in RT33 or RT33DR treated groups compared to the control group (FIG. 8B). Combined, these data indicate that both RT33 and RT33DR are well tolerated in vivo and can reduce tumor growth as single agents upon systemic administration.

### Discussion

Although a wide variety of vectors have been developed to deliver therapeutic agents across cellular membranes, CPPs have attracted considerable interest in the recent years for their unique translocation properties. The ability of CPPs to transport large molecular cargo in a plurality of cellular types with low toxicity have allowed the development of novel CPP-derived therapeutics against numerous disease, that have provided promising results in a number of preclinical and clinical studies (7).

Here, we identified and characterized a new CPP corresponding to residues 1177-1186 of human Acinus-L, termed hAP10, as well as its derivative hAP10DR. In vitro approaches demonstrated that hAP10 displayed excellent cell penetration efficiencies in both normal and cancerous cells, equaling classical CPPs such as TAT and penetratin while being among the shortest CPPs identified thus far. Previous studies have demonstrated that the guanidium group of arginine is critical for cationic CPPs activity, through interaction with negatively charged components of membranes, and the number of arginines present in a sequence affects internalization efficiency (32-34). Interestingly, we observed remarkably augmented cell penetration efficiency of the hAP10DR derivative, in which we replaced the negatively charged aspartic acid present in the wild type counterpart with an arginine, as hAP10DR largely outperformed hAP10 as well as TAT and penetratin. The cell penetration properties of CPPs is also dependent of their secondary structure and it has been shown that peptides with a α-helical region can more efficiently enter cells (35,36). hAP10 and hAP10DR mostly adopt a helical structure, which can therefore explain their interesting CPP properties. Importantly, neither hAP10 nor hAP10DR induced membrane disturbance or detectable cellular toxicity. Both peptides are also non-immunogenic, making them attractive and safe carriers for in vivo applications. CPPs internalization is widely accepted to involve energy-dependent endocytosis and/or direct translocation across biological membranes (7,37). Biochemical investigations revealed the involvement of a heparan sulfate proteoglycan-mediated micropinocytosis as a major route of internalization for hAP10 and hAP10DR. Still, as multi-endocytic routes are often involved in CPPs uptake, further studies would be needed to clarify the exact internalization mechanisms for hAP10 and hAP10DR. To further evaluating the potential of hAP10 and hAP10DR as macromolecules delivery tools, the peptides were firstly conjugated with GFP. Both hAP10-GFP and hAP10DR-GFP fusion proteins were efficiently transduced in cultured cells, demonstrating hAP10 and hAP10DR interest as novel vehicles for intracellular protein delivery. Of note, hAP10DR was a far better carrier than TAT or penetratin for GFP intracellular delivery, in lane with its superior penetrating ability. Finally, we evaluated the performances of hAP10 and hAP10DR through the design and study of tumor targeting peptides. Our previous studies showed that inhibiting interactions between the survival protein AAC-11 and its binding partners drastically increased susceptibility of tumor cells to apoptosis (23). Moreover, a cell penetrating peptide (peptide RT53) based on the fusion of the penetratine CPP and the heptad leucine repeat region of AAC-11 (residues 363-399), which functions as a protein-protein interaction module, was shown to induce cancer cell death in vitro and to inhibit melanoma tumor growth in a xenograft mouse model (30). We hypothesized here that a peptide similar to RT53 but based on hAP10 and hAP10DR CPPs might possess valuable anti-cancer properties. The heptad leucine repeat region of AAC-11 is encoded by two exons (exons 9 and 10). As exons often correspond to structural and functional units of a protein (38), one can envisioned that only one of the two exons encoding AAC-11 heptad leucine repeat region could carry the anticancer activity exhibited by the RT53 peptide, making it possible to shorten the AAC-11 specific domain of the peptide. Our previous work indicated that mutation of two exon 10-encoded leucine residues in RT53 (corresponding to positions 384 and 391 of AAC-11), identified as critical for AAC-11 scaffolding and anti-apoptotic function (23,39), abrogated RT53 anti-tumor activity (30). We therefore designed two peptides, designed RT33 and RT33DR, consisting of AAC-11 residues 377-399, that are encoded by exon 10, attached to the C-terminus of hAP10 or hAP10DR, respectively, and tested their anticancer properties. Interestingly, both peptides were able to selectively kill cancer cells in vitro, without affecting normal cells. RT33- and RT33DR-induced cancer cells death occurred through an apoptosis-independent, membranolytic mechanism, as evidenced by LDH release assays as well as electron microscopy results. Like RT53, RT33 and RT33DR accumulate at the plasma membrane level of cancer cells, but not of non-cancerous cells. Even known a contribution of the physico-chemical properties of tumor cells membranes cannot formally be excluded, we hypothesize that RT33 and RT33DR, as witnessed with other cancer cells specific, membrane active peptides (40-42), interact with a membrane partner(s) that is mainly expressed in the membrane of transformed cells. Upon binding, the helical structure of RT33 and RT33DR could allow the formation of pores in the cancer cell membrane, as observed with other membranolytic, pore forming peptides (43). Identification of RT33 and RT33DR membrane partner(s) is currently underway. The potential use of RT33 and RT33DR as novel anticancer drugs was then evaluated in the context of the Sézary Syndrom, a leukemic and aggressive form of cutaneous T cell lymphoma (CTCL) with poor prognosis. We chose to focus on Sézary Syndrom because current treatment options are limited, emphasizing the need for novel agents and therapeutic targets in these patients (44). Treatment of primary patient-derived samples with either RT33 or RT33DR, but not the hAP10 or hAP10DR shuttles alone, induced selective death of malignant T cell clone, while sparring the non-transformed T cell and the non-T cell populations. As observed with cancer cell lines, RT33 and RT33DR-induced Sézary cells death was necrotic, as validated by 7-AAD staining. In a xenograft model with HUT78 cells, systemic injection of RT33 and RT33DR resulted in significant reduction in tumor growth, confirmed by reduced tumor weight. Histological analysis of tumors derived from RT33 and RT33DR treated mice indicated increased necrotic cytotoxicity, compared to controls. In summary, we have developed novel, short, human-derived, non-cytotoxic and non-antigenic cell permeable peptides, showing excellent cell penetrating ability. Importantly, fusion peptides consisting of the survival protein AAC-11 residues 377-399 linked to the C-terminus of hAP10 or hAP10DR exhibited remarkable anticancer properties both ex vivo and in a mouse model of Sézary Syndrom. Therefore, we expect that the unique characteristics of hAP10 and hAP10DR will allow their use for a wide variety of in vitro and in vivo applications.

### REFERENCES

Throughout this application, various references describe the state of the art to which this invention pertains. The disclosures of these references are hereby incorporated by reference into the present disclosure.

1. Ye J, Liu E, Yu Z, Pei X, Chen S, Zhang P, et al. CPP-Assisted Intracellular Drug Delivery, What Is Next? Int J Mol Sci 2016;17(11).

2. Garnacho C. Intracellular Drug Delivery: Mechanisms for Cell Entry. Curr Pharm Des 2016;22(9):1210-26.

3. Cardoso A M, Trabulo S, Cardoso A L, Lorents A, Morais C M, Gomes P, et al. S4(13)-PV cell-penetrating peptide induces physical and morphological changes in membrane-mimetic lipid systems and cell membranes: implications for cell internalization. Biochimica et biophysica acta 2012;1818(3):877-88.

4. Alves I D, Goasdoue N, Correia I, Aubry S, Galanth C, Sagan S, et al. Membrane interaction and perturbation mechanisms induced by two cationic cell penetrating peptides with distinct charge distribution. Biochimica et biophysica acta 2008;1780(7-8):948-59.

5. Richard J P, Melikov K, Vives E, Ramos C, Verbeure B, Gait M J, et al. Cell-penetrating peptides. A reevaluation of the mechanism of cellular uptake. The Journal of biological chemistry 2003;278(1):585-90.

6. Maiolo J R, Ferrer M, Ottinger E A. Effects of cargo molecules on the cellular uptake of arginine-rich cell-penetrating peptides. Biochimica et biophysica acta 2005;1712(2):161-72.

7. Guidotti G, Brambilla L, Rossi D. Cell-Penetrating Peptides: From Basic Research to Clinics. Trends Pharmacol Sci 2017;38(4):406-24.

8. Bechara C, Sagan S. Cell-penetrating peptides: 20 years later, where do we stand? FEBS letters 2013;587(12):1693-702.

9. Johnson R M, Harrison S D, Maclean D. Therapeutic applications of cell-penetrating peptides. Methods in molecular biology 2011;683:535-51.

10. de la Fuente J M, Berry C C. Tat peptide as an efficient molecule to translocate gold nanoparticles into the cell nucleus. Bioconjug Chem 2005;16(5):1176-80.

11. Zatsepin T S, Turner J J, Oretskaya T S, Gait M J. Conjugates of oligonucleotides and analogues with cell penetrating peptides as gene silencing agents. Curr Pharm Des 2005;11(28):3639-54.

12. Liu H, Zeng F, Zhang M, Huang F, Wang J, Guo J, et al. Emerging landscape of cell penetrating peptide in reprogramming and gene editing. Journal of controlled release: official journal of the Controlled Release Society 2016;226:124-37.

13. Suresh B, Ramakrishna S, Kim H. Cell-Penetrating Peptide-Mediated Delivery of Cas9 Protein and Guide RNA for Genome Editing. Methods in molecular biology 2017;1507:81-94.

14. Bullok K E, Dyszlewski M, Prior J L, Pica C M, Sharma V, Piwnica-Worms D. Characterization of novel histidine-tagged Tat-peptide complexes dual-labeled with (99 m) Tc-tricarbonyl and fluorescein for scintigraphy and fluorescence microscopy. Bioconjug Chem 2002; 13(6): 1226-37.

15. Nguyen Q T, Olson E S, Aguilera T A, Jiang T, Scadeng M, Ellies L G, et al. Surgery with molecular fluorescence imaging using activatable cell-penetrating peptides decreases residual cancer and improves survival. Proceedings of the National Academy of Sciences of the United States of America 2010;107(9):4317-22.

16. Olson E S, Jiang T, Aguilera T A, Nguyen Q T, Ellies L G, Scadeng M, et al. Activatable cell penetrating peptides linked to nanoparticles as dual probes for in vivo fluorescence and MR imaging of proteases. Proceedings of the National Academy of Sciences of the United States of America 2010;107(9):4311-6.

17. De Groot A S, Scott D W. Immunogenicity of protein therapeutics. Trends Immunol 2007;28(11):482-90.

18. Chauhan A, Tikoo A, Kapur A K, Singh M. The taming of the cell penetrating domain of the HIV Tat: myths and realities. Journal of controlled release: official journal of the Controlled Release Society 2007;117(2):148-62.

19. Vucetic Z, Zhang Z, Zhao J, Wang F, Soprano K J, Soprano D R. Acinus-S′ represses retinoic acid receptor (RAR)-regulated gene expression through interaction with the B domains of RARs. Molecular and cellular biology 2008;28(8):2549-58.

20. Rodor J, Pan Q, Blencowe B J, Eyras E, Caceres J F. The RNA-binding profile of Acinus, a peripheral component of the exon junction complex, reveals its role in splicing regulation. RNA 2016;22(9): 1411-26.

21. Tange T O, Shibuya T, Jurica M S, Moore M J. Biochemical analysis of the EJC reveals two new factors and a stable tetrameric protein core. RNA 2005;11(12):1869-83.

22. Joselin A P, Schulze-Osthoff K, Schwerk C. Loss of Acinus inhibits oligonucleosomal DNA fragmentation but not chromatin condensation during apoptosis. The Journal of biological chemistry 2006;281(18): 12475-84.

23. Rigou P, Piddubnyak V, Faye A, Rain J C, Michel L, Calvo F, et al. The antiapoptotic protein AAC-11 interacts with and regulates Acinus-mediated DNA fragmentation. The EMBO journal 2009;28(11):1576-88.

24. Schwerk C, Prasad J, Degenhardt K, Erdjument-Bromage H, White E, Tempst P, et al. ASAP, a novel protein complex involved in RNA processing and apoptosis. Molecular and cellular biology 2003;23(8):2981-90.

25. Gautam A, Chaudhary K, Kumar R, Sharma A, Kapoor P, Tyagi A, et al. In silico approaches for designing highly effective cell penetrating peptides. J Transl Med 2013;11:74.

26. Futaki S. Membrane-permeable arginine-rich peptides and the translocation mechanisms. Advanced drug delivery reviews 2005;57(4):547-58.

27. Eiriksdottir E, Konate K, Langel U, Divita G, Deshayes S. Secondary structure of cell-penetrating peptides controls membrane interaction and insertion. Biochimica et biophysica acta 2010;1798(6):1119-28.

28. Buchan D W, Minneci F, Nugent T C, Bryson K, Jones D T. Scalable web services for the PSIPRED Protein Analysis Workbench. Nucleic acids research 2013;41(Web Server issue):W349-57.

29. Roy A, Kucukural A, Zhang Y. I-TASSER: a unified platform for automated protein structure and function prediction. Nat Protoc 2010;5(4):725-38.

30. Jagot-Lacoussiere L, Kotula E, Villoutreix B O, Bruzzoni-Giovanelli H, Poyet J L. A Cell-Penetrating Peptide Targeting AAC-11 Specifically Induces Cancer Cells Death. Cancer research 2016;76(18):5479-90.

31. Kohnken R, Fabbro S, Hastings J, Porcu P, Mishra A. Sezary Syndrome: Clinical and Biological Aspects. Curr Hematol Malig Rep 2016;11(6):468-79.

32. Amand H L, Rydberg H A, Fornander L H, Lincoln P, Norden B, Esbjorner E K. Cell surface binding and uptake of arginine- and lysine-rich penetratin peptides in absence and presence of proteoglycans. Biochimica et biophysica acta 2012;1818(11):2669-78.

33. Zhang D, Wang J, Xu D. Cell-penetrating peptides as noninvasive transmembrane vectors for the development of novel multifunctional drug-delivery systems. Journal of controlled release: official journal of the Controlled Release Society 2016;229:130-9.

34. Wender P A, Mitchell D J, Pattabiraman K, Pelkey E T, Steinman L, Rothbard J B. The design, synthesis, and evaluation of molecules that enable or enhance cellular uptake: peptoid molecular transporters. Proceedings of the National Academy of Sciences of the United States of America 2000;97(24):13003-8.

35. Park C B, Yi K S, Matsuzaki K, Kim M S, Kim S C. Structure-activity analysis of buforin II, a histone H2A-derived antimicrobial peptide: the proline hinge is responsible for the cell-penetrating ability of buforin II. Proceedings of the National Academy of Sciences of the United States of America 2000;97(15):8245-50.

36. Krautwald S, Dewitz C, Fandrich F, Kunzendorf U. Inhibition of regulated cell death by cell-penetrating peptides. Cell Mol Life Sci 2016;73(11-12):2269-84.

37. Patel L N, Zaro J L, Shen W C. Cell penetrating peptides: intracellular pathways and pharmaceutical perspectives. Pharmaceutical research 2007;24(11):1977-92.

38. Go M. Correlation of DNA exonic regions with protein structural units in haemoglobin. Nature 1981;291(5810):90-2.

39. Tewari M, Yu M, Ross B, Dean C, Giordano A, Rubin R. AAC-11, a novel cDNA that inhibits apoptosis after growth factor withdrawal. Cancer research 1997;57(18):4063-9.

40. Do T N, Rosal R V, Drew L, Raffo A J, Michl J, Pincus M R, et al. Preferential induction of necrosis in human breast cancer cells by a p53 peptide derived from the MDM2 binding site. Oncogene 2003;22(10):1431-44.

41. Kanovsky M, Raffo A, Drew L, Rosal R, Do T, Friedman F K, et al. Peptides from the amino terminal mdm-2-binding domain of p53, designed from conformational analysis, are selectively cytotoxic to transformed cells. Proceedings of the National Academy of Sciences of the United States of America 2001;98(22):12438-43.

42. Sarafraz-Yazdi E, Bowne W B, Adler V, Sookraj K A, Wu V, Shteyler V, et al. Anticancer peptide PNC-27 adopts an HDM-2-binding conformation and kills cancer cells by binding to HDM-2 in their membranes. Proceedings of the National Academy of Sciences of the United States of America 2010;107(5):1918-23.

43. Polyansky A A, Chugunov A O, Vassilevski A A, Grishin E V, Efremov R G. Recent advances in computational modeling of alpha-helical membrane-active peptides. Current protein & peptide science 2012;13(7):644-57.

44. Hughes C F, Khot A, McCormack C, Lade S, Westerman D A, Twigger R, et al. Lack of durable disease control with chemotherapy for mycosis fungoides and Sezary syndrome: a comparative study of systemic therapy. Blood 2015;125(1):71-81.

1. A peptide that consists of the amino acid sequence as set forth in SEQ ID NO:1 (RSRSR-X6-RRRK), wherein X6 is D or R.

2. The peptide of claim 1 that consists of the amino acid sequence as set forth in SEQ ID NO:2 (RSRSRDRRRK) or SEQ ID NO:3 (RSRSRRRRRK).

3. (canceled)

4. A method of transporting a cargo moiety to a subcellular location of a cell, the method comprising contacting the cell with the cargo moiety covalently linked to the peptide of claim 1 for a time sufficient for allowing the peptide to translocate the cargo moiety to the subcellular location.

5. A complex wherein the peptide of claim 1 is covalently linked to a cargo moiety.

6. The complex of claim 5 wherein the cargo moiety is selected from the group consisting of carbohydrate, lipid, nucleic acid, peptide, polypeptide, protein, bacteriophage or virus particle, synthetic polymer, resin, latex particle, dye and other detectable molecules.

7. The complex of claim 5 wherein the peptide is fused to at least one heterologous polypeptide so as to form a fusion protein.

8. The complex of claim 7 wherein the at least one heterologous polypeptide is a fluorescent protein.

9. The complex of claim 7 wherein the at least one heterologous polypeptide is a cancer therapeutic polypeptide.

10. The complex of claim 7 wherein the peptide is fused to:
an amino acid sequence ranging from the phenylalanine residue at position 380 to the leucine residue at position 384 in SEQ ID NO:4 or,
an amino acid sequence ranging from the phenylalanine residue at position 380 to the isoleucine residue at position 388 in SEQ ID NO:4 or,
an amino acid sequence ranging from the phenylalanine residue at position 380 to the leucine residue at position 391 in SEQ ID NO:4 or,
an amino acid sequence ranging from the tyrosine residue at position 379 to the leucine residue at position 391 in SEQ ID NO:4 or,
an amino acid sequence ranging from the glutamine residue at position 378 to the leucine residue at position 391 in SEQ ID NO:4 or,
an amino acid sequence ranging from the leucine residue at position 377 to the leucine residue at position 391 in SEQ ID NO:4 or,
an amino acid sequence ranging from the lysine residue at position 371 to the glycine residue at position 397 in SEQ ID NO:4 or,
an amino acid sequence ranging from the lysine residue at position 371 to the leucine residue at position 391 in SEQ ID NO:4 or,
an amino acid sequence ranging from the phenylalanine residue at position 380 to the threonine residue at position 399 in SEQ ID NO:4 or,
an amino acid sequence ranging from the lysine residue at position 371 to the threonine residue at position 399 in SEQ ID NO:4 or,
an amino acid sequence ranging from the leucine residue at position 377 to the threonine residue at position 399 in SEQ ID NO:4.

11. The complex of claim 10 wherein the fusion protein consists of the amino acid sequence as set forth in SEQ ID NO:5 (RSRSRDRRRKLQYFARGLQVYIRQLRLALQGKT) or SEQ ID NO:6 (RSRSRRRRRKLQYFARGLQVYIRQLRLALQGKT).

12. A method of treating a disease in a subject in need thereof comprising administering to the subject a therapeutically effective amount of the complex of claim 5.

13. The method of claim 12 wherein the disease is cancer.

14. The method of claim 13 wherein the cancer is Sezary syndrome.

15. A pharmaceutical composition comprising the complex of claim 5 combined with pharmaceutically acceptable excipients.


SEQUENCE LISTING (6 sequences)

SEQ ID NO: 1 - protein, 10 aa, Artificial
  Synthetic cell penetrating peptide
  MISC_FEATURE (6)..(6): X is D or R
RSRSRXRRRK

SEQ ID NO: 2 - protein, 10 aa, Artificial
  Synthetic hAP10 cell penetrating peptide
RSRSRDRRRK

SEQ ID NO: 3 - protein, 10 aa, Artificial
  Synthetic hAP10DR cell penetrating peptide
RSRSRRRRRK

SEQ ID NO: 4 - protein, 524 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQKVEIGQKRASEDTTSGSPPKKSSAGPKRDARQIYNPPSGKYSSNLGNFNYEQRGAFRGSRGGRGWGTRGNRSRGRLY

SEQ ID NO: 5 - protein, 33 aa, Artificial
  Synthetic RT-33 fusion protein
RSRSRDRRRKLQYFARGLQVYIRQLRLALQGKT

SEQ ID NO: 6 - protein, 33 aa, Artificial
  Synthetic RT-33DR fusion protein
RSRSRRRRRKLQYFARGLQVYIRQLRLALQGKT